(19) United States
(12) Patent Application Publication
(10) Pub. No.: US 2012/0226134 A1
(43) Pub. Date: Sep. 6, 2012

(54) SYSTEM AND METHOD FOR CONTROLLING THERAPY MACHINE

(75) Inventors: Sang Gyu Ju, Seoul (KR); Youngyih Han, Seoul (KR); Yong Chan Ahn, Seoul (KR); Jin Sung Kim, Yongin-si (KR)

(73) Assignee: SAMSUNG LIFE WELFARE FOUNDATION, Seoul (KR)

(21) Appl. No.: 13/509,323

(22) PCT Filed: Nov. 12, 2009
(86) PCT No.: PCT/KR2009/006655

§ 371 Date: May 11, 2012

(51) Int. Cl. A61B 6/00

(52) U.S. Cl. 600/407

(57) ABSTRACT

A system for controlling a therapy machine is a method for controlling a therapy machine for irradiating radiation onto a patient whose body has been stabilized. The method acquires a video signal constituted by a plurality of pixels including information on the posture of the patient; converts the video signal to digital format, and generates video data including information on characteristic values of each of the plurality of pixels; and calculates, on the basis of the information, the number of pixels from among the plurality of pixels of which the characteristic values lie outside a predetermined range. Then, the therapy machine is controlled based on the calculated number. Thus, medical accidents due to inattention during observation by therapists may be prevented, and the accuracy of therapy through the therapy machine may be improved.

### CROSS-REFERENCE TO RELATED APPLICATION

This application claims priority to and the benefit of Korean Patent Application No. 10-2008-0057199 filed in the Korean Intellectual Property Office on Jun. 18, 2009, the entire contents of which are incorporated herein by reference.

### BACKGROUND OF THE INVENTION

(a) Field of the Invention

The present invention relates to a system and method for controlling a radiation therapy machine. More particularly, the present invention relates to a system and method for controlling a radiation therapy machine based on the movement of a treated patient.

(b) Description of the Related Art

When a patient is being treated with a medical therapy machine, movement by the patient can lead to a medical accident. In particular, when radiation is irradiated on a patient through a radiation therapy machine, in order to minimize detrimental effects on normal tissue of the patient and focus the irradiation of radiation on tumor, movement of the patient should be restricted during the irradiation of radiation.

To this end, therapists in the related art have used various tools to stabilize the body of a patient, and have monitored the patient through a video or by direct observation.

However, there is the problem that during the course of treatment over a prolonged period, a medical accident can arise such as improperly irradiated radiation on a patient by patient movement or falling of the patient due to the oversight in patient monitoring of a therapist.

The above information disclosed in this Background section is only for enhancement of understanding of the background of the invention and therefore it may contain information that does not form the prior art that is already known in this country to a person of ordinary skill in the art.

### SUMMARY OF THE INVENTION

The present invention has been made in an effort to provide a system and method for controlling a therapy machine having the advantages of preventing a medical accident from occurring due to the patient movement during radiation therapy and oversight in patient monitoring by therapist for a medical therapy machine, and enabling accurate irradiation of radiation.

An exemplary embodiment of the present invention provides a method for controlling a therapy machine that irradiates radiation onto a patient, whose body is stabilized, the method including: acquiring a video signal including information on a posture of the patient and including a plurality of pixels including a plurality of pixels containing information on a posture of the patient; converting the video signal to digital format, and generating video data including a characteristic value of each of the plurality of pixels; calculating, based on the information, the number of pixels from among the plurality of pixels, the characteristic values of which lie outside a predetermined range; and controlling the therapy machine based on the number.

Another exemplary embodiment of the present invention provides a method for controlling a therapy machine for stabilizing the body of a patient and performing therapy, the method including: receiving a therapy start signal from the therapy machine; acquiring a reference video signal including a plurality of pixels containing information on a posture of the patient, according to the therapy start signal; acquiring a monitoring video signal including a plurality of pixels containing information on the posture of the patient after the reference video signal is acquired; and controlling the therapy machine on the bases of the reference video signal and the monitoring video signal.

Yet another exemplary embodiment of the present invention provides a system for controlling a therapy machine for stabilizing the body of a patient and performing therapy, the system including: a video acquisition unit, a video analysis unit, and a control unit. The video acquisition unit is for acquiring a video signal including a plurality of pixels containing information on a posture or movement of the patient whose body is stabilized on the therapy machine, the video analysis unit is for analyzing the video signal and generating analysis results; and the control unit is for controlling the therapy machine according to the analysis results.

The system and method for controlling a therapy machine according to exemplary embodiments of the present invention may monitor the posture or movements of a patient, who receives therapy from a therapy machine, through a video signal of a video capturing the patient, so as to prevent a medical accident from occurring due to inattention during video monitoring by a therapist, and to improve the accuracy of treatment through the therapy machine. Further, the work efficiency of a therapist may be improved by using a network to integrate and manage a plurality of therapy machines.

### BRIEF DESCRIPTION OF THE DRAWINGS

FIG. 1 is a block diagram illustrating a system for controlling a therapy machine according to an exemplary embodiment of the present invention.

FIG. 2 is a block diagram illustrating a video analysis unit according to an exemplary embodiment of the present invention.

FIG. 3 is a flowchart illustrating a method for controlling a therapy machine according to an exemplary embodiment of the present invention.

FIG. 4 is a drawing illustrating an integrated management system of a therapy machine according to an exemplary embodiment of the present invention.

### DETAILED DESCRIPTION OF THE EMBODIMENTS

In the following detailed description, only certain exemplary embodiments of the present invention have been shown and described, simply by way of illustration. As those skilled in the art would realize, the described embodiments may be modified in various different ways, all without departing from the spirit or scope of the present invention. The drawings and description are to be regarded as illustrative in nature and not restrictive. Like reference numerals designate like elements throughout the specification.

Throughout the specification, unless explicitly described to the contrary, the word “comprise” and variations such as “comprises” or “comprising”, will be understood to imply the inclusion of stated elements but not the exclusion of any other elements. Further, the terms “-er”, “-or” and “module” described in the specification mean units for processing at least one function and operation and can be implemented by hardware components or software components and combinations thereof.

Hereinafter, a system and method for controlling a therapy machine according to an exemplary embodiment of the present invention will be described in detail with reference to the drawings.

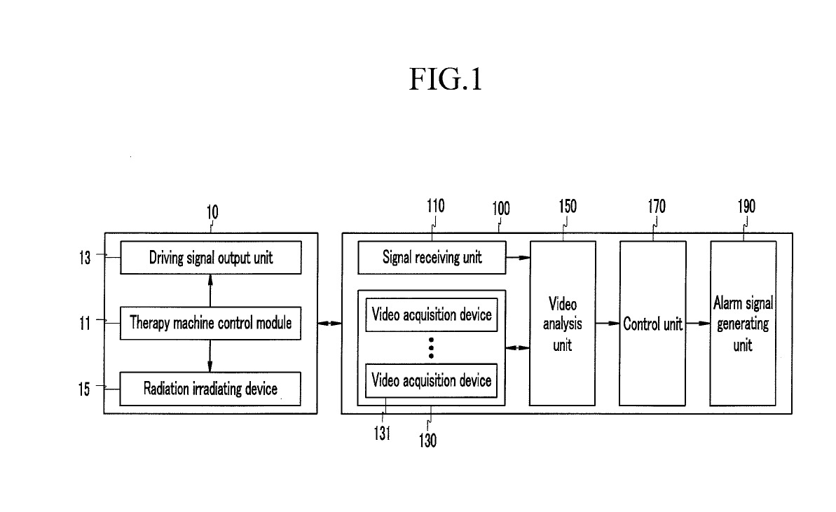

First, with reference to FIG. 1, a system for controlling a therapy machine according to an exemplary embodiment of the present invention will be described.

FIG. 1 is a block diagram illustrating a system for controlling a therapy machine according to an exemplary embodiment of the present invention.

As shown in FIG. 1, a system 100 for controlling a therapy machine according to an exemplary embodiment of the present invention monitors the movements of a patient receiving radiation therapy through a radiation therapy machine 10, and controls the radiation therapy machine 10.

The radiation therapy machine 10 includes a therapy control module 11, a driving signal output unit 13, and a radiation irradiating device 15.

The therapy machine control module 11 forms an interface with a therapist to receive control commands and control the radiation irradiating device 15 according to the received control commands. Here, the therapy machine control module 11 may receive control commands from a therapy machine control system 100. Further, the therapy machine control module 11 may transfer control information corresponding to received control commands to the driving signal output unit 13. In this case, the control information may include radiation irradiation start information or radiation irradiation end information.

The driving signal output unit 13 receives control information from the therapy machine control module 11, and transmits the radiation irradiating start signal or the radiation irradiating end signal according to the transferred control information to the therapy machine control system 100.

The radiation irradiation device 15 irradiates radiation onto a patient according to the controlling by the therapy machine control module 11.

The therapy machine control system 100 includes a signal receiving unit 110, a video acquisition unit 130, a video analysis unit 150, a control unit 170, and an alarm signal generating unit 190.

The signal receiving unit 110 receives a signal transferred from the radiation therapy machine 10, and transfers the received signal to the video analysis unit 150.

The video acquisition unit 130 acquires a video signal, including information on the posture or movement of a patient including a plurality of pixels containing information on a posture of the patient whose body is made stationary through the radiation therapy machine 10 in order to receive radiation therapy. In this case, the video acquisition unit 130 may include a plurality of video acquisition devices 131 installed in different positions, and may capture the patient from different positions through the plurality of video acquisition devices 131 and acquire a plurality of video signals respectively corresponding to the plurality of video acquisition devices 131. Further, the plurality of video acquisition devices 131 may be directly installed on the radiation irradiating device 15 of the radiation therapy machine 10, or may be installed proximate to the radiation irradiating device 15.

The video analysis unit 150 controls the video acquisition unit 130 on the basis of a signal relayed from the signal receiving unit 110, analyzes the video signal acquired by the video acquisition unit 130, and transfers the analysis results to the control unit 170. In this case, the video analysis unit 150 may analyze each of a plurality of pixels constituting the acquired video signal, and detect a boundary of an object projected on the acquired video signal, so as to analyze the video signal. Further, the video analysis unit 150 may analyze the acquired video signal through various video analysis algorithms.

When it is determined that there has been a change in the posture of the patient on the basis of the analysis results transferred from the video analysis unit 150, the control unit 170 controls the therapy machine control module 11 through a control signal, and controls the alarm signal generating unit 190 through an alarm command.

The alarm signal generating unit 190 generates an alarm signal according to the alarm command from the control unit 170, to signal that a movement of the patient has been detected to the therapist operating the radiation therapy machine 10. In this case, while not illustrated in FIG. 1, the alarm signal generating unit 190 may include a lamp or alarm sound generator, and the alarm signal may be generated through the lamp or alarm sound generator.

Hereinafter, in order to clarify the description of a system and method for controlling a therapy machine according to an exemplary embodiment of the present invention, the reference numerals illustrated in FIG. 1 may be cited.

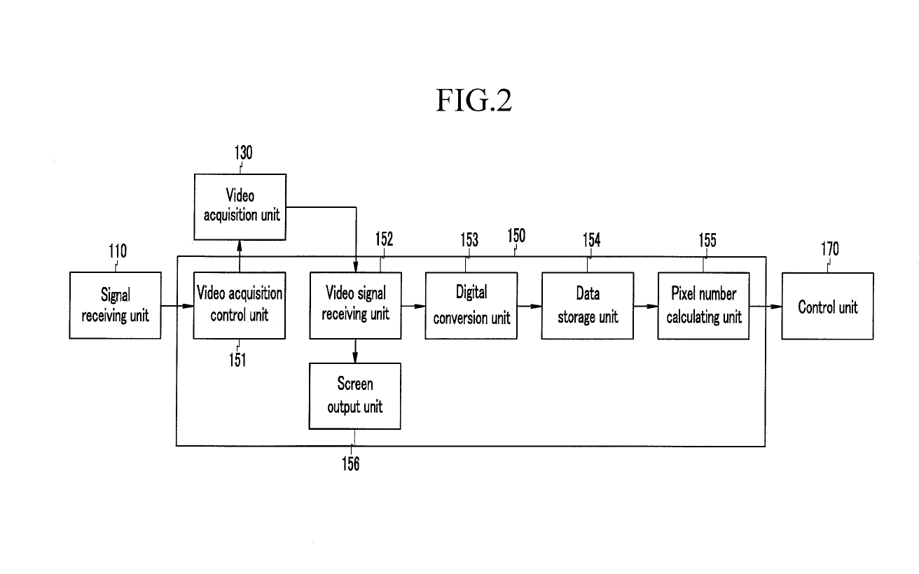

Next, referring to FIG. 2, a video analysis unit of a therapy machine control system according to an exemplary embodiment of the present invention will be described.

FIG. 2 is a block diagram illustrating a video analysis unit according to an exemplary embodiment of the present invention.

As illustrated in FIG. 2, a video analysis unit 150 according to an exemplary embodiment of the present invention includes a video acquisition control unit 151, a video signal receiving unit 152, a digital conversion unit 153, a data storage unit 154, a pixel number calculation unit 155, and a screen output unit 156.

The video acquisition control unit 151 receives a signal from the signal receiving unit 110, and controls the video acquisition unit 130 according to the received signal. In this case, the video acquisition control unit 151 may control the video acquisition unit 130 so that the video acquisition unit 130 acquires a video signal at certain time intervals.

The video signal receiving unit 152 receives a video signal from the video acquisition unit 130.

The digital conversion unit 153 converts a video signal to digital form to generate video data corresponding to the video signal, and the video data generated by the digital conversion unit 153 includes information on each characteristic value of a plurality of pixels (hereinafter, referred to as “characteristic value information”). In this case, according to a method of mixing the red, green, and blue for each pixel in a plurality of pixels and representing the colors red-green-blue (hereinafter referred to as ‘RGB’), the characteristic values of the plurality of pixels respectively include ratio values of the red, green, and blue. Further, according to a black and white method of mixing the black and white for each pixel in a plurality of pixels and representing colors, the characteristic values of the plurality of pixels respectively include ratio values of black and white.

The data storage unit 154 stores video data. In this case, the data storage unit 154 may store a plurality of video data.

The pixel number calculation unit 155 calculates the number of pixels that lie outside a predetermined range of characteristic values from among a plurality of pixels, on the basis of characteristic value information included in the video data, generates information on the calculated number of pixels, and transfers the generated information to the control unit 170.

The screen output unit 156 outputs a video corresponding to a video signal received by the video signal receiving unit 152. In this case, the therapist may monitor the posture or movement of the patient through the video output through the screen output unit 156.

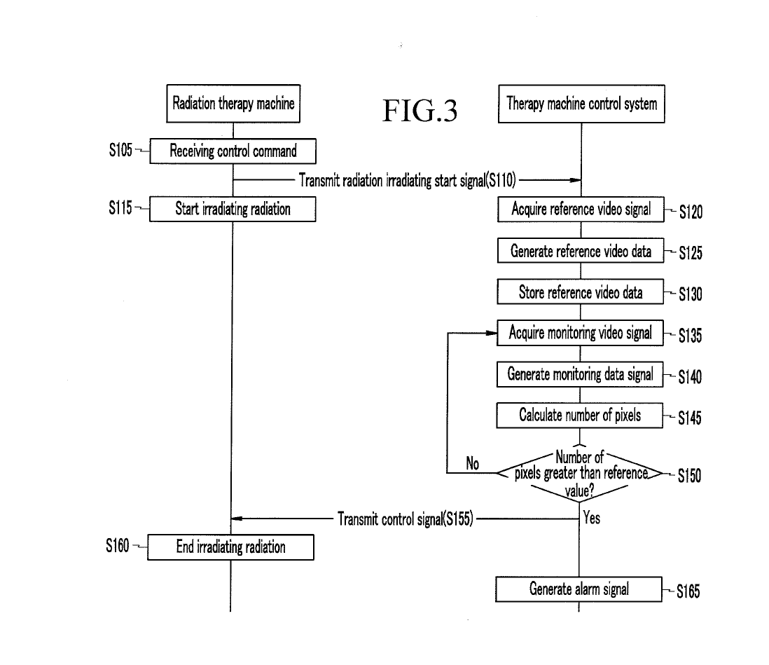

Next, referring to FIG. 3, a method using a system to control a radiation therapy machine according to an exemplary embodiment of the present invention will be described.

FIG. 3 is a flowchart illustrating a method for controlling a therapy machine according to an exemplary embodiment of the present invention.

As illustrated in FIG. 3, first, the therapy machine control module 11 receives a control command in step S105 for operating the radiation therapy machine 10. In this case, after the therapist operating the radiation therapy machine 10 makes the body of the patient stationary, the therapist may interface with the therapy machine control module 11 to transmit a control command to the therapy machine control module 11.

Next, the driving signal output unit 13 transmits a radiation irradiating start signal to the therapy machine control system 100 in step S110. In this case, the therapy machine control module 11 may transfer radiation irradiating start information corresponding to the received control command to the driving signal output unit 13, and the driving signal output unit 13 may transmit a radiation irradiating start signal corresponding to the transferred radiation irradiating start information to the therapy machine control system 100.

Then, the radiation irradiating device 15 begins irradiating radiation on the patient in accordance with the controlling by the therapy machine control module 11 in step S115.

The video acquisition control unit 151 of the video analysis unit 150 controls the video acquisition unit 130 according to the radiation irradiating start signal, and the video acquisition unit 130 acquires a reference video signal constituted of a plurality of pixels including information on the posture of the patient according to the controlling by the video acquisition control unit 151, in step S120. In this case, the signal receiving unit 110 receives the radiation irradiating start signal and transfers the radiation irradiating start signal to the video acquisition control unit 151, and the video acquisition control unit 151 controls the video acquisition unit 130 according to the transferred radiation irradiating start signal.

Next, the video signal receiving unit 152 of the video analysis unit 150 receives a reference video signal from the video acquisition unit 130, and the digital conversion unit 153 of the video analysis unit 150 converts the reference video signal to digital format and generates reference video data corresponding to the reference video signal, in step S125. In this case, the reference video data includes characteristic value information.

Then, the data storage unit 154 of the video analysis unit 150 stores the reference video data in step S130.

Next, the video acquisition control unit 151 of the video analysis unit 150 controls the video acquisition unit 130 so that the video acquisition unit 130 acquires a video signal at certain time intervals, and the video acquisition unit 130 acquires a video signal in step S135 including information on the posture of the patient at a first time and configured with a plurality of pixels, according to the controlling by the video acquisition control unit 151. In this case, the first time corresponds to a certain time that has elapsed after the video acquisition unit 130 has acquired the video signal. Further, the time interval in which the video acquisition unit 130 acquires the video signal may be predetermined, and may be received as an input from the therapist.

Next, when the video signal receiving unit 152 of the video analysis unit 150 receives a monitoring video signal from the video acquisition unit 130, the digital conversion unit 153 of the video analysis unit 150 converts the monitoring video signal into digital format and generates monitoring video data corresponding to the monitoring video signal in step S140. In this case, the monitoring video data include characteristic value information.

Then, the pixel number calculating unit 155 of the video analysis unit 150, on the basis of reference video data and monitoring video data, calculates a difference value between each characteristic value of a plurality of pixels of the monitoring video signal and each character value of a plurality of pixels of the reference video signal, and calculates the number of pixels for(of?) which the difference value exceeds a threshold value, from among the plurality of pixels of the monitoring video signal, in step S145.

Next, the control unit 170 determines in step S150 if the number of pixels calculated by the pixel number calculating unit 155 exceeds a predetermined reference value.

If the number of pixels exceeds the reference value, the control unit 170 determines that a change has occurred in the posture of the patient and transmits a control signal including a control command for ending the irradiation of radiation to the therapy machine control module 11, in step S155.

Next, the therapy machine control module 11 controls the radiation irradiating device 15 according to the control command included in the control signal, and the radiation irradiating device 15 ends the irradiation of radiation on the patient in step S160, in accordance with the controlling by the therapy machine control module 11.

Meanwhile, the control unit 170 controls the alarm signal generating unit 190 through an alarm command for alerting the therapist that a change has occurred in the posture of the patient, and the alarm signal generating unit 190 generates an alarm signal through a lamp or alarm sound generator, according to the alarm command from the control unit 170, in step S165.

Meanwhile, if the number of pixels does not exceed the reference value, the monitoring video signal acquiring step S135 is performed again to acquire a monitoring video signal, and then the steps following the monitoring video signal acquiring step S135 are performed. In this case, the video acquisition unit 130 may acquire a monitoring video signal including information on the posture of the patient constituted of a plurality of pixels including information on the posture of the patient at a second time and constituted of a plurality of pixels, and here, the second time may correspond to a certain time that has elapsed after the first time.

In this case, FIG. 3 illustrates the processes up to the process in which the therapy machine control system 100 stops radiation irradiation by the radiation therapy machine 10. However, during the irradiation of radiation by the radiation therapy machine 10, the therapist may stop radiation irradiation through a control command for a therapist to directly stop radiation irradiation, or the irradiation of radiation on the patient may be ended. In this case, the driving signal output unit 13 transmits a radiation irradiating stop signal to the therapy machine control system 100, the therapy machine control system 100 stops the monitoring of the patient in accordance with the radiation irradiating stop signal, and a standby for signal state is maintained until a radiation irradiating start signal is received.

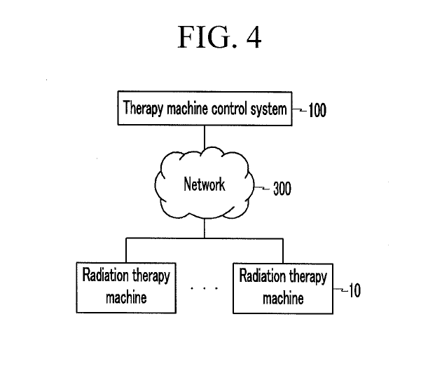

Next, with reference to FIG. 4, a therapy machine integrated management system that uses a therapy machine control system according to an exemplary embodiment of the present invention will be described.

FIG. 4 is a drawing illustrating an integrated management system of a therapy machine according to an exemplary embodiment of the present invention.

As illustrated in FIG. 4, a therapy machine integrated control system according to an exemplary embodiment of the present invention includes a therapy machine control system 100, a network 300, and a plurality of radiation therapy machines 10.

The therapy machine control system 100 is connected to a plurality of radiation therapy machines 10 through the network 300, and controls each of the radiation therapy machines 10.

The network 300 is a communication network that links the therapy machine control system 100 and the plurality of radiation therapy machines 10. In this case, the network 300 may allocate an address for each of the plurality of radiation therapy machines 10, and may notify the therapy machine control system 100 of the allocated addresses.

Each of the plurality of radiation therapy machines 10 is a piece of medical equipment that performs radiation therapy by irradiating a patient with radiation according to the controlling by the therapy machine control system 100.

The objects to be controlled by the above-described system and method for controlling a therapy machine according to an exemplary embodiment of the present invention are not limited to radiation therapy machines, and the system and method for controlling a therapy machine may be employed for various types of unmanned medical equipment.

The above-mentioned exemplary embodiments of the present invention are not embodied only by an apparatus and method. Alternatively, the above-mentioned exemplary embodiments may be embodied by a program performing functions, which correspond to the configuration of the exemplary embodiments of the present invention, or a recording medium on which the program is recorded. These embodiments can be easily devised from the description of the above-mentioned exemplary embodiments by those skilled in the art to which the present invention pertains.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

What is claimed is:

1. A method for controlling a therapy machine that irradiates radiation onto a patient whose body is stabilized, the method comprising:
acquiring a video signal comprising information on a posture of the patient and comprising a plurality of pixels;
converting the video signal to digital format, and generating video data comprising information on a characteristic value of each of the plurality of pixels;
calculating, based on the information, a number of pixels from among the plurality of pixels, of which the characteristic values lie outside a predetermined range; and
controlling the therapy machine based on the number.

2. The method for controlling a therapy machine of claim 1, wherein:
the controlling includes
determining whether the number exceeds a predetermined reference value; and
transmitting a control signal to the therapy machine when the number exceeds the reference value.

3. The method for controlling a therapy machine of claim 2, wherein
the video signal comprises information on the posture of the patient at a first time, and
the acquiring includes
acquiring a video signal comprising information on the posture of the patient at a second time when the number does not exceed the reference value.

4. The method for controlling a therapy machine of claim 2, further comprising:
generating an alarm signal when the number the exceeds the reference value.

5. A method for controlling a therapy machine for stabilizing the body of a patient and performing therapy, the method comprising:
receiving a therapy start signal from the therapy machine;
acquiring a reference video signal comprising a plurality of pixels containing information on a posture of the patient, according to the therapy start signal;
acquiring a monitoring video signal comprising a plurality of pixels containing information on the posture of the patient after the reference video signal is acquired; and
controlling the therapy machine on the bases of the reference video signal and the monitoring video signal.

6. The method for controlling a therapy machine of claim 5, further comprising:
converting the reference video signal to digital format, and generating reference video data comprising information on a characteristic value of each of the plurality of pixels; and
converting the monitoring video signal to digital format, and generating monitoring video data comprising information on a characteristic value of each of the plurality of pixels, wherein
the controlling includes
controlling the therapy machine on the bases of the reference video data and the monitoring video data.

7. The method for controlling a therapy machine of claim 6, wherein;
the controlling the therapy machine on the bases of the reference video data and the monitoring video data includes
calculating, on the bases of the reference video data and the monitoring video data, difference values between the characteristic values of each of the plurality of pixels of the monitoring video signal and the characteristic values of each of the plurality of pixels of the reference video signal, and calculating a number of pixels, from among the plurality of pixels of the monitoring video signal, of which the difference values exceed a predetermined threshold value; and
controlling the therapy machine based on the number.

8. The method for controlling a therapy machine of claim 7, wherein
the controlling the therapy machine on the bases of the reference video data and the monitoring video data further includes
determining whether the number exceeds a predetermined reference value; and
transmitting a control signal to the therapy machine when the number exceeds the reference value.

9. The method for controlling a therapy machine of claim 8, wherein
the monitoring video signal includes information on the posture of the patient at a first time, and
the acquiring the monitoring video signal includes
acquiring a monitoring video signal including a plurality of pixels containing information on the posture of the patient at a second time, when the number does not exceed the reference value.

10. The method for controlling a therapy machine of claim 8, wherein
the controlling the therapy machine on the bases of the reference video data and the monitoring video data further includes
generating an alarm signal when the number exceeds the reference value.

11. A system for controlling a therapy machine for stabilizing the body of a patient and performing therapy, the system comprising:
a video acquisition unit for acquiring a video signal including a plurality of pixels containing information on a posture or movement of the patient whose body is stabilized on the therapy machine;
a video analysis unit for analyzing the video signal and generating analysis results; and
a control unit for controlling the therapy machine according to the analysis results.

12. The system for controlling a therapy machine of claim 11, wherein
the video analysis unit includes
a digital conversion unit for converting the video signal to digital format, and generating video data including information on a characteristic value of each of the plurality of pixels; and
a pixel number calculating unit for calculating, on the basis of the video data, a number of pixels, from among the plurality of pixels constituting the video signal, of which characteristic values lie outside a predetermined range.

13. The system for controlling a therapy machine of claim 12, wherein
the characteristic value includes
a ratio value for each of red, green and blue.

14. The system for controlling a therapy machine of claim 12, wherein
the characteristic value includes
a ratio value for black and for white.

15. The system for controlling a therapy machine of claim 12, wherein
the control unit
controls the therapy machine based on the number of the pixels.

16. The system for controlling a therapy machine of claim 11, wherein
the video acquisition unit includes
a video acquisition device installed directly on the therapy machine or installed proximate to the therapy machine.

17. The system for controlling a therapy machine of claim 11, further comprising
an alarm signal generating unit for generating an alarm signal according to the controlling by the control unit.
